(19) United States
(12) Patent Application Publication
(10) Pub. No.: US 2013/0018006 A1
(43) Pub. Date: Jan. 17, 2013

(54) Atheroscleroclastic Bioceutical Formulations

(71) Applicant: Stephen King, Kempner, TX (US)

(72) Inventor: Stephen King, Kempner, TX (US)

(21) Appl. No.: 13/440,985

(22) Filed: Apr. 5, 2012

Related U.S. Application Data
(60) Provisional application No. 61/572,336, filed on Jul. 14, 2011.

(51) Int. Cl. A61K 31/7028; A61K 31/685; A61P 9/10

(52) U.S. Cl. 514/25; 514/77

(57) ABSTRACT

A group of compounds formed from phosphorylated monosaccharides and/or phosphorylated oligosaccharides esterified with physiologic methyl donors consisting of choline, and methyl donors chosen from a group consisting of esteried with a substance chosen from the group consisting of methylene choline and all their derivatives and metabolites thereof.

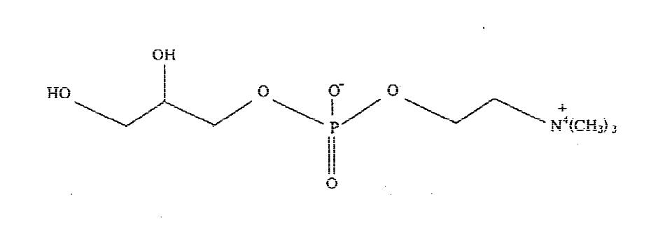

A new compound, glycerylphosphorylcholine (GPC), and its use for slowing or reducing athersclerosis and arthritis, especially osteoarthritis. Several classes of agents to prevent and cure hardening of the arteries is described. The same physiologic interaction of calcium and phosphorus applies throughout. Additional components also make it effective against rheumatoid arthritis.

This application claims priority of Provisional Application No. 61/572,336 filed on Jul. 14, 2011 by Stephen King, PhD

### BACKGROUND OF THE INVENTION

Arteriosclerosis includes all forms of gradual diminution of arterial structure and function, a.k.a. hardening of the arteries. Atherosclerosis is occlusion of arteries by deposits of matter; for the most part, in arteries of large diameter.

Quite distinct in its mechanism of action, intrinsic arteriosclerosis occurs as the arterial wall itself becomes stiff and brittle like an old rubber hose left outdoors and microscopically degraded under the action of inclement weather. Its causes include cross-linking of collagen, weakening of the smooth muscle layer, cumulative sequelae of transient hypoxia, and even some degree of heavy metal poisoning.

Arteriolar sclerosis is hardening of small arteries, the arterioles. This begins with intrinsic stiffening of the arteriole but may progress with plaque formation also.

As atherosclerosis is diminished by the action of the hydrophilic lecithinoids herein disclosed, the arterial wall itself is newly exposed to corrective factors within the blood, chiefly antioxidants such as vitamin C, vitamin E, and SOD (superoxide dismutase). Since the antioxidant strength of blood is variable, some embodiments of this invention are augmented with materials chosen for such effect.

The sum total of these mechanisms of action amounts to substantial rejuvenation of the arterial tree and, to some extent, the heart itself.

Atherosclerosis and arteriolar sclerosis cause many cases of chronic ischemia, cerebrovascular pathology, cardiac failure, and various sequelae thereof. Risk factors and countermeasures thereto have been studied for centuries, if not millennia. Diet, exercise, chronic and acute stress, and even some iatrogenic factors have been implicated. Modalities such as chelation, hyperbaric oxygen, and meganutrients have all been employed for countervailing effects. Even so, this class of pathologies accounts for a significant fraction of human deaths in the civilized world.

This invention is founded on compositions of matter to mitigate this pathology with a class of antisclerotic pharmaceutical and nutriceutical agents designed for maximum health with minimum harm. Arterial plaque consists of multiple substances and generally is best met with multiple therapeutic effects.

In general, the function of an artery is obstructed by excess matter in its lumen and by stiffening and weakening of the matter comprising it. Embodiments of this invention, or sclerolytics, correct the former, though some also address the latter.

The essential trace element phosphorus occurs in DNA, RNA, ATP, GDP, the phosphorylated metabolic intermediates along the physiologic pathways of glycolysis and oxidative metabolism, phosphorylated buffer systems of intracellular and extracellular fluids, in lecithins and cephalins, active sites of some enzymes, et al.

In the energy metabolism of aerobic cells quite generally, fructose-1,6-diaphosphate (hereinafter FDP) is made, used, and rapidly replaced. It and its metabolites glyceraldehyde 3-phosphate, dihydroxyacetone phosphate, et al., provide steady energy flow, some of the precursors for biosynthesis of endogenous lecithins and cephalins, support for protein metabolism, functional immunoglobin pathways, biosynthesis of nucleotide coenzymes and neurotransmitters, and, indirectly, the nucleic acids themselves.

It stands to reason that the production, distribution, and metabolism of FDP is generally maintained on an even keel but might be stressed during hypoglycemic episodes, chronically recurrent prediabetic and outright diabetic dysfunction, chronic and acute ischemia, alcohol excess, and the requirements of tissue repair from time to time.

One can even out the FDP supply with enzymatic release from esters such as that shown in “The FIGURE”, by various enzymes such as the buterylcholinesterase. One can also reasonably expect to enhance FDP persistence and availability by formulating that substrate with some of its natural metabolites like glycerophosphorylcholine, thus slowing catabolism of FDP esters, FDP itself, and various metabolites thereof, until physiologic requirements increase at specific sites during trauma, ischemia, etc. These products can be used to help repair and function of circulatory, visceral, endocrine, and neurologic systems. Some such formulations, suitably screened, may enhance obstetric and pediatric care. Of special interest in medical, dental, and veterinary contexts are those biochemical and physiologic effect on deposits of calcium, fat, and fibrin in the circulatory system.

Physiologic effects of products of this invention mobilize fat-soluble deposits from tissues generally and from the vascular tree in particular. When fats are compounded with calcium or complexed with calcium salts, not excluding calcium carbonate, they are deposited, microscopically, colloidally, and macroscopically, throughout the body. Physiologic effects, of lipotropic factors in general and products of this invention in particular, mobilize fat-soluble deposits from tissues generally and from the vasculature in particular. Glycerophosphorylcholine (GPC) and choline itself are naturally occurring lipotropic factors similar to products of this invention.

In this context gallstones present an interesting case. They consist largely of calcium bilirubinate, but with appreciable cholesterol and other fatty matter. As calcium is leached out by the phosphate, fatty matter is gradually exposed to lipotropic effects and processes. While fatty acids are most easily taken up forming lecithin, cholesterol and similar hydrophobic materials are taken up into the microscopic fat globules emulsified with the blood and lymph. Indeed, bilirubin and biliary agents akin to it are excellent emulsifiers. When gallstones are present, products of this invention cause their calcium and fats to be largely secreted into the bile duct, go into the small intestine, and affect the composition of the blood. The paradoxical result is that cholesterol, bilirubin, and other constituents of the gallstones may appear at increased levels in the clinical chemistry.

Something similar happens in the case of kidney stones, but the mechanism of action is different. The calcareous component is much greater, and the lipid content is negligible. Again, however, the action of phosphate on calcium results in a soluble product. In this case, however, relatively little calcium is taken to storage in the long bones: most is simply excreted. Since urinary calculi grow at times of excess calcium in circulation, this calcium excretion may well be of benefit.

Aside from its role in building bone, calcium at the cell membrane, in physiologic balance with magnesium and phosphorus, helps maintain proper electrochemical charge distribution. Calcium deficiency can cause painful cramps in the skeletal muscles and impede the normal contractions of arteries and the heart itself. For this reason, inter alia, one sometimes sees anomalous cardiac failure not explained by the degree of arterial occlusion. On the other hand, calcium leached out of the plaque becomes available for smooth operation of the chemical machinery of muscular contraction.

### DISCLOSURE OF THE PROCESS

Fructose-1,6-diphosphate is a natural product occurring in the Embden-Meyerhof pathway leading from glucose absorption to the Krebs cycle. It is one of several products formulated by incubating crystalline fructose with phosphoric acid (30% H₃PO₄ in aquo works nicely) and separating it by column chromatography; other syntheses have been reported in the literature.

One can esterify FDP with methionine, choline, carnitine, serylcholine, DMAE, or any other methyl donor of normal cellular metabolism. For most uses contemplated here, one choline onto each phosphate gives a perfectly satisfactory result. Persistence of these, particularly P, P′-dicholyl-FDP, can be extended with their simpler metabolites, particularly GPC (=glycerylphosphorylcholine). The latter combines with fatty acids by action of lecithin synthestase.

Practical mixtures of GPC with P,P′-dicholyl-FDP—hereinafter F(PCh)₂—also supply phosphate, a necessary nutrient that combines with calcium to form crystals stored in the long bones; in just the same way, it removes calcium deposits from arthritic joints, depositing that calcium in the long bones also and freeing the cartilage of the joint for physiologic restoration. As calcium and fat are mobilized from arterial plaque, the fat can be burned off or simply excreted, while the calcium itself is also stored or excreted. Removal of these materials gradually exposes fibrin (the binding material in blood clots) to the action of circulating proteolytic enzymes. When plaque is forming, calcified fat (like soapscum in hard water) and fibrin (the essential structural protein of blood clots) gather, narrowing and stiffening the arterioles, starving the soft tissues of nutrients and oxygen to an extent, also increasing levels of carbon dioxide therein. F(PCh)₂ and GPC work to dissolve this plaque, moving arterial patency closer to optimal. Digestion of fibrin by circulating enzymes reinforces this effect; benign atheroma cells can then be attacked, clearing the arterial tree still further.

Arteries harden not only by plaque formation but by stiffening of the arterial wall itself. For the most part, this happens because collagen becomes cross-linked, like an old rubber hose exposed to outdoor weather for a long time; it becomes not only stiff but brittle. In extreme cases, these phenomena contribute to heart attacks, cerebral vascular accidents (strokes), thrombosis, compromised vision and hearing, etc. As plaque is removed, circulating superoxide dismutase from hepatic secretions and some foods gradually unstiffens the collagen, replacing it with newly synthesized material; the exact mechanisms for this effect were unknown a few years ago and may still be unknown. In time, however, the arteries unharden.

The optimal proportions of F(PCh)₂ and GPC are not predictable from armchair theorizing (not by present company, anyway!) but could be determined by resorting to well-designed experiments on suitable animal models.

### PREFERRED EMBODIMENT

Combine fructose and glycerin in aqueous solution; 20 gm fructose to 80 gm glycerin is preferred.

Phosphorylcholine is formed by proportional combination of phosphoric acid with choline chloride. This is heated by any suitable means, such as Bunsen burner or microwave diathermy, in enough water to dissolve both.

The two solutions are then mixed so that each mole of glycerin is provided at least one mole of phosphorylcholine, while each mole of fructose is provided at least two. These are to be stirred together and heated to something in the range of 50° C. to 60° C. or until the whole appears uniformly mixed. The totality is evaporated to dryness, then dissolved in hot water (about 80° C.), allowed to cool, then, filtered. The filtrate can be redissolved in more hot water.) This gives a saturated solution at room temperature; this can then be refrigerated to about 5° C. or 40° F.) and, if necessary, filtered again.

This cold solution can be diluted for bottling. One liter of solution added to 9 liters of water gives the 10% solution; this is diluted 50:50 in more water to give the 5% solution which is for sale to the public.

### BRIEF DESCRIPTION OF THE DRAWING

Chemical structure of GPC, a natural product related to embodiments of this invention, as specified in The FIGURE. Glycerin is esterified with phosphoric acid; phosphoric acid is further esterified with choline. This phosphodiester is the same as lecithin itself with both fatty acyl moieties removed. Its titer is raised by metabolism of products of this invention.

What is claimed is:

1. A compound comprising a monosaccharide esterified with at least one molar equivalent of phosphorylcholine (excluding, however the case of glycerin itself esterified with only one molar equivalent of phosphorylcholine and not otherwise modified) and all esters, amides, thioesters, or salts with minerals and/or biogenic amines, as dissolved in a solvent system of at least one hydrophilic compound whether solid or as dissolved in any solid system comprising at least one hydrophilic compound.

2. All esters, amides, thioesters, or salts with biogenic amines of glycerin esterified with one molar equivalent of phosphorylcholine, as dissolved in a solvent system of at least one hydrophilic compound whether solid or as dissolved in any solid system comprising at least one hydrophilic compound.

3. The compound of claim 1 or 2 derivatized with any amino acid, an oligopeptide of fewer than 22 amino acids, any nucleotide or any oligonucleotide of fewer than 22 units, whether from ribose or deoxyribose.

4. The compound of claim 1, 2 or 3 wherein at least one choline is replaced by at least one other physiologic methyl donor, suspended, dissolved emulsified or mixed in the same way.

5. Any compound of claim 1, 2 or 3 where the molar equivalent of phosphorylcholine is dicholyl-FDP, or dicholyl-dRPD, where dRPD is deoxyribose 3,5-diphosphate.

6. The compound of claim 1, wherein the hydrophilic compound of the solvent system is glyceraldehyde 3-phosphate, its choline ester, glycerin, or water.

7. The compound of claim 1, or 2 derivatized with a vitamin, whether solid or dissolved in any solvent system of claim 1.

8. The compound of claim 7 wherein the solvent system is a cysteinyl methionyl ester of dicholyl glucose 1,6-diphosphate in aqueous 20% ethyl acetate (aq), or dicholyl glucose 1,6-diphosphate in an aqueous solution of ethyl acetate.

9. The compound of claim 3, wherein the physiologic methyl donor is dimethylaminoethanol.

10. The compound of claim 4, wherein the suspension, emulsion, or colloid is dicholyl glyceryl diphosphate in aqueous gelatin.

11. A new use of the compound of claim 3 or 4 to slow or reverse arteriosclerosis or arthritis.
